(19) United States
(12) Patent Application Publication
(10) Pub. No.: US 2012/0330432 A1
(43) Pub. Date: Dec. 27, 2012

(54) FINGER PROSTHESIS

(75) Inventor: Tik-Pui Daniel Fong, Hong Kong (CN)

(73) Assignee: THE CHINESE UNIVERSITY OF HONG KONG, Hong Kong (CN)

(21) Appl. No.: 13/529,762

(22) Filed: Jun. 21, 2012

Related U.S. Application Data
(60) Provisional application No. 61/499,695, filed on Jun. 22, 2011.

(51) Int. Cl. A61F 2/42

(52) U.S. Cl. 623/21.15

(57) ABSTRACT

A finger prosthesis comprising a tip for simulating different functions of a finger and a ring for attaching the prosthesis to a stump. The finger prosthesis further includes a joint coupled between the tip and the ring, wherein the joint includes an interlocking mechanism configured to allow the joint to rotate at lockable intervals so as to lock the tip into different positions during a flexion period of the finger prosthesis.

### CROSS REFERENCE TO RELATED APPLICATIONS

This application claims the benefit of priority under 35 U.S.C. § 119(e) to U.S. Provisional Patent Application No. 61/499,695 filed on Jun. 22, 2011, which is explicitly incorporated by reference in its entirety.

### BACKGROUND

Hand function is very important to a human's daily life. Finger loss leads to weakened hand function, medical burden, diminished working capability and inferior quality of life. “X-finger”, the world's first active-function artificial finger, has been invented for patients to regain the finger function. Such finger allows users to perform flexion and extension actively. However, due to the completed structure, this kind of finger is expensive and is often not affordable to most people, especially to those low-income factory workers who lost their fingers due to occupational accidents. The current proposal aims to design an inexpensive artificial finger.

### SUMMARY

In one aspect of the present application, a finger prosthesis is provided. The finger prosthesis comprises a tip for simulating different functions of a finger and a ring for attaching the prosthesis to a stump. The finger prosthesis further comprises a joint coupled between the tip and the ring, wherein the joint comprises an interlocking mechanism configured to allow the joint to rotate at lockable intervals so as to lock the tip into different positions during a flexion period of the finger prosthesis.

In another aspect of the present application, there is provided a finger prosthesis comprising: a tip for simulating different functions of a finger; and a ring for attaching the prosthesis to a stump. The finger prosthesis further comprises a joint coupled between the tip and the ring, wherein the joint comprises a shifting part with a first engaging end; and a rotating wheel with a second engaging end, wherein the second engaging end is capable of being interlocked with the first engaging end by a plurality of intervals.

According to one embodiment, the rotating part may be provided with a rebounding mechanism configured to make the rotating wheel to return to a start position at the end of the intervals.

In another aspect of the present application, there is provided a finger prosthesis comprising: a tip for simulating different functions of a finger; a ring for attaching the prosthesis to a stump; and a joint coupled between the tip and the ring. The joint comprises:
a shifting part with a first plurality of gears; and
a rotating wheel with a second plurality of gears, wherein the shifting part is capable of being interlocked with the rotating wheel through the first and second gears by a plurality of intervals;
a capacity for receiving the shifting part;
a support located in the capacity for supporting one side of the shifting part to provide a spring-like nature that allows little displacement of the shifting part;
wherein the rotating wheel is provided with a first protuberance and a second protuberance located two sides of the second gears, and wherein, during an interlocked stage, the shifting part is located with one side of the capacity, at the end of the intervals, the first protuberance exerts a force on an end of the shifting part so that the shifting part is shifted to the other side of the capacity, allowing the rotating wheel to return to a start position.

### BRIEF DESCRIPTION OF THE DRAWINGS

FIG. 1 illustrates the finger prosthesis according to one embodiment of the present application.

FIG. 2 is a decomposition view for the finger prosthesis as shown in FIG. 1.

FIG. 3 illustrates the inner configurations of the joint according to one embodiment of the present application.

FIG. 4(a)-(e) illustrate the operation principle of the joint.

FIG. 5 illustrates flexion operation of the finger prosthesis according to one embodiment of the present application.

FIG. 6 illustrates six lockable intervals of the joint according to one embodiment of the present application.

### DETAILED DESCRIPTION OF THE PREFERRED EMBODIMENTS

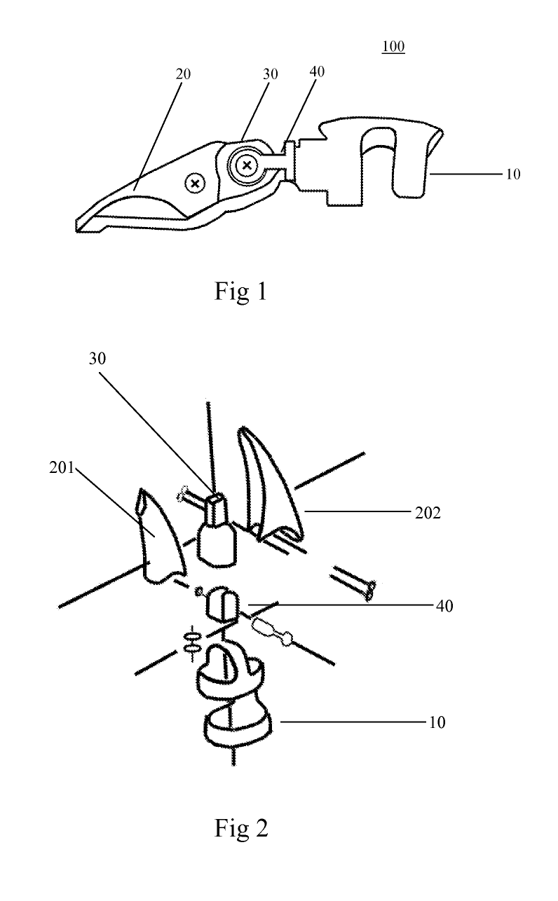

FIG. 1 illustrates the finger prosthesis 100 according to one embodiment of the present application. As shown, the finger prosthesis 100 comprises a ring 10 for attaching the prosthesis to a stump (not shown), and a tip 20 configured to simulate different functions of a finger. There is a joint 30 coupled between the tip 20 and the ring 10. Inside the joint 30, there is configured with an interlocking mechanism for allowing the joint to rotate at lockable intervals (for example, there may be six intervals) so as to lock the tip 20 into different positions during a flexion period of the finger prosthesis 100.

According to one embodiment, the ring 10 has a non-invasive ring like structure to attach the prosthesis 100 to the stump of the metacarpal bone surrounding with muscles and soft tissue. The ring 10 may be made of soft metal, for example sliver, that allows easy and fine adjustment with manual strength.

As shown in FIG. 2, the tip 20 may comprise an posterior portion 201 and a anterior portion 202 opposite to the posterior portion 201. The portions 201 and 202 may be made from silver metal and silicon, respectively. In order to better apply friction to the held object, silicon was used to form the posterior portion 201 of the tip, since it can be shaped easily by molding and is slightly deformable to maximize the contact area while holding object. The anterior portion 202 of the tip was made of silver plate which simulates the nail function in real finger for fine motion.

The joint 30 may be connected to the ring 10 through a connector 40. The connector 40 may also function to adjust the length of the finger prosthesis 100. That is, the more length the connector 40 has, the more length the finger prosthesis 100 will have.

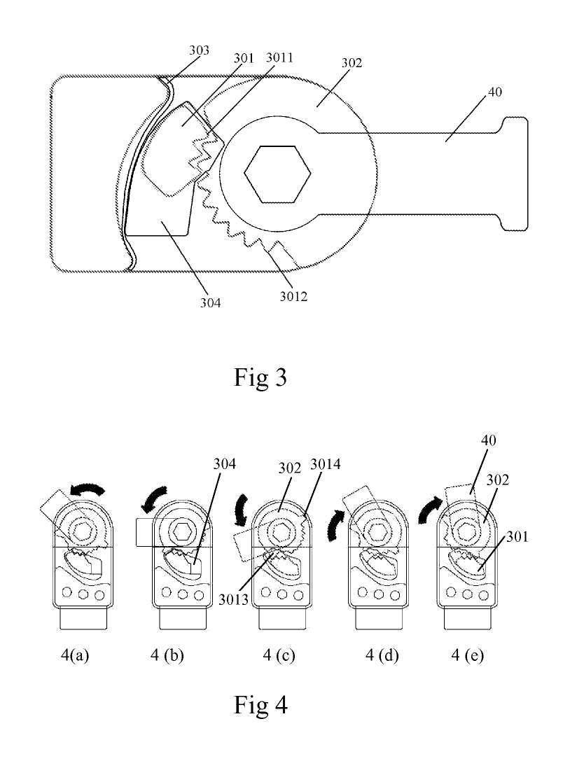

In one embodiment, the interlocking mechanism in the joint 30 may comprise a rebounded type progressive hinge lock which is bent easily in one direction and locked against an opposite force. The rebounded type progressive hinge lock is often used in chairs, seats and sofas for adjusting the back support angle. The rebounded type progressive hinge lock may comprise a shifting part with a first engaging end, and a rotating part with a second engaging end. The second engaging end is capable of being interlocked with the first engaging end by a plurality of intervals of the flexion period. The rotating part may be further provided with a rebounding mechanism, which makes the rotating part to return to the start position smoothly at the end of the sixth intervals. A more specific example of the interlocking mechanism is given as below in reference to FIG. 3.

FIG. 3 illustrates the elements insides the joint 30 with the interlocking mechanism according to a specific example of the present application. In this example, the interlocking mechanism may comprise a shifting part 301, a rotating wheel 302 and a supporting part 303.

As shown in FIG. 3, there is provided a capacity 304 in the joint 30 for receiving the shifting part 301. During the flexion period, the shifting part 301 and the rotating wheel 302 may be interlocked by gears 3011 and 3012 at, for example, six intervals, and the shifting part is backed with the supporting part 303, which provides a spring-like nature that allows little displacement of the shifting part. The supporting part 303 may be of a thin sheet, for example, a thin aluminum sheet.

In addition, the rotating wheel 302 is further provided with two protuberances 3013 and 3014, which cooperate with the other elements of the interlocking mechanism to function as the rebounding mechanism, which will be discussed below.

Referring to FIG. 4 (a), the shifting part 301 and the rotating wheel 302 may be interlocked by gears 3011 and 3012. As the connector 40 rotates according to the direction of arrow, which in turn makes the rotating wheel 302 to rotate in the same direction, the other side of the shifting part 301 is backed with the supporting part 303. In the FIGS. 4(c) and (d), at the end of the intervals, the protuberance 3013 of the rotating wheel 302 exerts force on the end of the shifting part 301 and then the shifting part 301 would be shifted to the other side of the capacity 304, allowing the rotating wheel 302 to return to the start position smoothly. In FIG. 4 (e), as the rotating wheel 302 further rotates, the protuberance 3014 of the rotating wheel would push the shifting part 301 back to the “locking” position. In one embodiment, all moving parts in the joint may be made of stainless steel for longer duration while aluminum was chosen for other parts for weight reduction.

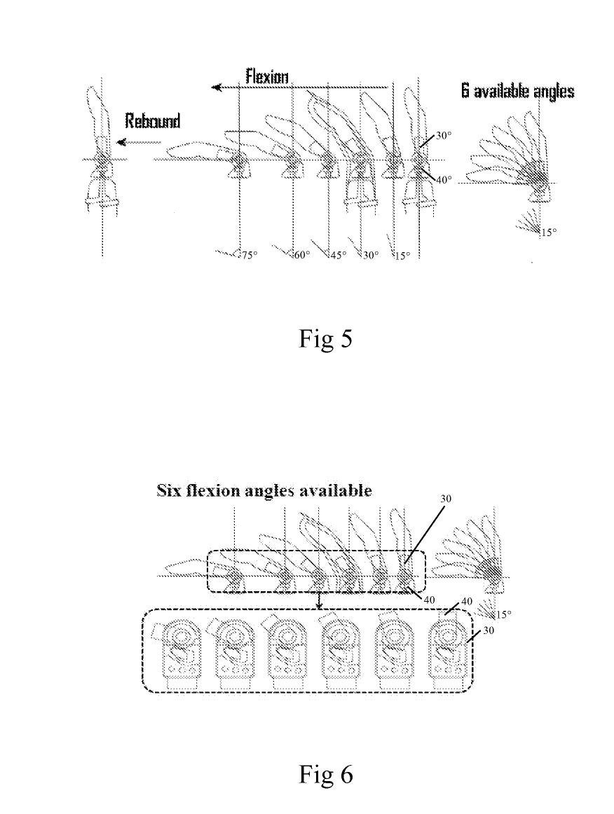

FIG. 5 illustrates the different flexion status of the prosthesis according to one embodiment of the present application. FIG. 6 illustrates the relationship between each flexion status and the joint. In this embodiment, there are six intervals during the flexion. As shown, the joint starts working from zero flexion and then flexion is performed by assistance of another hand, with 15 degrees increment, until a 90-degree flexion is reached. At each of the intervals, the joint is locked to prevent extension of finger. This makes the thumb opposition nature of human hand for amputee become possible. When the flexion angle exceeds 90-degree, it rebounds and returns to starting position. In addition, the flexion is done by assistance of another hand or any rigid surface. At each of the angles, the shifting part and the rotating part gets locked by the gears to prevent extension, thus providing a rigid support for hand grip. To reset the device to full extension, just flex it to 90 degrees and it gets rebounded to the original position.

The finger prosthesis allows user to regain flexion and extension movement of finger. The prosthesis can restore the opposition function of patient with multiple digit amputation. By applying enough prosthesis on the involved hand, it is possible for them to pick up large heavy object.

While the present application has been illustrated by the above description and embodiments or implementations, it is not intended to restrict or in any way limit the scope of the appended claims hereto.

What is claimed is:

1. A finger prosthesis comprising:
a tip for simulating different functions of a finger;
a ring for attaching the prosthesis to a stump; and
a joint coupled between the tip and the ring, wherein the joint comprises an interlocking mechanism configured to allow the joint to rotate at lockable intervals so as to lock the tip into different positions during a flexion period of the finger prosthesis.

2. The prosthesis of claim 1, wherein the interlocking mechanism comprises a rebounded type progressive hinge lock, which is capable of being bent in a first direction and locked against an force with a second direction, the second direction being substantively opposite to the first direction.

3. The prosthesis of claim 2, wherein the rebounded type progressive hinge lock comprises:
a shifting part with a first engaging end; and
a rotating part with a second engaging end, wherein the second engaging end is capable of being interlocked with the first engaging end by a plurality of intervals.

4. The prosthesis of claim 3, wherein each of the first and second engaging ends comprises a plurality of gears, and the second engaging end is capable of being interlocked with the first engaging end through the gears.

5. The prosthesis of claim 3, wherein the rotating part is provided with a rebounding mechanism configured to make the rotating part to return to a start position at the end of the intervals.

6. The prosthesis of claim 5, wherein rebounding mechanism comprises two protuberances on the rotating part.

7. The prosthesis of claim 3, wherein the joint comprises a capacity for receiving the shifting part.

8. The prosthesis of claim 7, wherein during an interlocked stage, the shifting part is located in one side of the capacity.

9. The prosthesis of claim 8, wherein, at the end of the intervals, the rebounding mechanism exerts a force on an end of the shifting part so that the shifting part is shifted to the other side of the capacity, allowing the rotating part to return to a start position.

10. The prosthesis of claim 7, wherein there is provided a support part in the capacity for supporting one side of the shifting part to provide a spring-like nature that allows little displacement of the shifting part.

11. The prosthesis of claim 10, wherein the support part comprises a sheet-like shape.

12. The prosthesis of claim 3, wherein the shifting part and rotating part are configured to cooperate to make the two parts interlocked by 6 intervals.

13. The prosthesis of claim 1, wherein the ring comprises a non-invasive ring like structure to attach the prosthesis to the stump.

14. The prosthesis of claim 1, wherein the tip comprises an anterior portion and a posterior portion opposite to the anterior portion, the anterior portion and the posterior portion are made from silver metal and silicon, respectively, in order to simulate different functions of the finger.

15. A finger prosthesis comprising:
a tip for simulating different functions of a finger;
a ring for attaching the prosthesis to a stump; and
a joint coupled between the tip and the ring, wherein the joint comprises:
a shifting part with a first engaging end; and
a rotating wheel with a second engaging end, wherein the second engaging end is capable of being interlocked with the first engaging end by a plurality of intervals.

16. The prosthesis of claim 15, wherein the rotating wheel is provided with a rebounding mechanism configured to make the rotating wheel to return to a start position at the end of the intervals.

17. The finger prosthesis comprising:
a tip for simulating different functions of a finger;
a ring for attaching the prosthesis to a stump; and
a joint coupled between the tip and the ring, wherein the joint comprises:
a shifting part with a first plurality of gears; and
a rotating wheel with a second plurality of gears, wherein the shifting part is capable of being interlocked with the rotating wheel through the first and second gears by a plurality of intervals;
a capacity for receiving the shifting part;
a support located in the capacity for supporting one side of the shifting part to provide a spring-like nature that allows little displacement of the shifting part;
wherein the rotating wheel is provided with a first protuberance and a second protuberance located two sides of the second gears,
wherein, during an interlocked stage, the shifting part is located in one side of the capacity, at the end of the plurality of intervals, the first protuberance exerts a force on an end of the shifting part so that the shifting part is shifted to the other side of the capacity, allowing the rotating wheel to return to a start position.
